(19) United States
(12) Patent Application Publication
(10) Pub. No.: US 2020/0376288 A1
(43) Pub. Date: Dec. 3, 2020

(54) TRAUMATIC TISSUE INJURY TREATMENT SYSTEMS

(71) Applicant: WetWare Biosystems LLC, Hilton, NY (US)

(72) Inventor: Bradley Richard Smith, Ellicott City, MD (US)

(73) Assignee: WetWare Biosystems LLC, Hilton, NY (US)

(21) Appl. No.: 16/888,193

(22) Filed: May 29, 2020

Related U.S. Application Data
(60) Provisional application No. 62/854,965, filed on May 30, 2019.

(51) Int. Cl. A61N 2/00; A61N 2/02

(52) U.S. Cl. CPC A61N 2/006; A61B 2090/065; A61N 2/02

(57) ABSTRACT

A wearable article can include one or more sensors configured to sense one or more of pressure, force, acceleration, and/or tissue activity. The wearable article can also include one or more stimulators configured to generate a magnetic field and positioned to apply the magnetic field to a tissue of a user, i.e., when worn, for treating the tissue after a predetermined pressure, force, acceleration, and/or tissue activity is sensed by the one or more sensors.

### CROSS-REFERENCE TO RELATED APPLICATIONS

This application claims priority to and the benefit of U.S. Provisional Application No. 62/854,965, filed May 30, 2019, the entire contents of which are herein incorporated by reference in their entirety.

### FIELD

This disclosure relates to traumatic tissue injury treatment systems.

### BACKGROUND

There are over three million cases of Traumatic Brain Injury (TBI) in the United States alone, for example. Each year, these TBIs cause over $86 billion in annual healthcare costs. After a TBI occurs, brain lesions expand rapidly within one hour. Thus this initial period after the injury is a critical, and traditionally unreachable, treatment window. When left untreated, more than two million neurons die per minute after a severe TBI, which can cause lifelong disability.

Such conventional methods and systems have generally been considered satisfactory for their intended purpose. However, there is still a need in the art for improved TBI treatment systems, for example. The present disclosure provides a solution for this need.

### SUMMARY

A wearable article can include one or more sensors configured to sense one or more of pressure, force, acceleration, and/or tissue activity. The wearable article can also include one or more stimulators configured to generate a magnetic field and positioned to apply the magnetic field to a tissue of a user, i.e., when worn, for treating the tissue after a predetermined pressure, force, acceleration, and/or tissue activity is sensed by the one or more sensors.

In certain embodiments, each of the one or more stimulators can be configured to apply a respective magnetic field to brain tissue to prevent propagation of a traumatic brain injury in the brain tissue. For example, in certain embodiments, the wearable article can be or include helmet padding, for example. The one or more stimulators can be disposed in the helmet padding and configured to be positioned to apply the magnetic field to one or more predetermined areas of a brain, for example. In certain embodiments, the wearable article can include a hard shell, and the helmet padding with the one or more stimulators can be disposed within the helmet shell, for example. Any other suitable wearable article having any suitable components and/or configured for any suitable body part(s) is contemplated herein.

In certain embodiments, the one or more stimulators can each include one or more coils. The one or more coils can be positioned such that a central axis of the one or more coils can be substantially perpendicular to a scalp surface when the wearable device is worn. In certain embodiments, the one or more stimulators can be configured to generate a magnetic field having a strength of about 0.5 Tesla (e.g., about 0.5 to about 1 Tesla) or above. Any other suitable stimulator (e.g., configured to produce a suitable electric field and/or magnetic field) is contemplated herein.

The one or more stimulators can include at least one frontal lobe stimulator and at least one (e.g., two symmetrically opposing) parietal and/or temporal stimulator (e.g., positioned to affect both the parietal and temporal lobe), for example. Any other suitable stimulator(s) for any suitable locations (e.g., occipital lobe, any suitable lobe boundaries) are contemplated herein.

In certain embodiments, the wearable article can include a controller operatively connected to the one or more sensors and configured to determine when a shock event indicative of a traumatic brain injury occurs. In certain embodiments, the controller can be configured to activate an indicator when the shock event occurs. For example, the indicator can be or include an LED. Any other suitable indicator (e.g., visual, invisible, audible, tactile, etc.) are contemplated herein.

In certain embodiments, the stimulators and/or the controller and/or the one or more sensors can be configured to connect to an external power supply (e.g., one or more batteries, capacitors, power electronics, control modules, etc.) via an input. The external power supply can be separate from the helmet and configured to provide suitable energy to generate each respective magnetic field.

In certain embodiments, however, a power source (e.g., one or more batteries and/or capacitors) can be connected to and/or contained within the helmet. The controller can be configured to cause energy from the power source to flow to the one or more stimulators to cause generation of each magnetic field.

For example, the controller can be configured to pulse each stimulator to create a pulsed magnetic field. The controller can be configured to provide one or more pulses to each stimulator at a repeating rate of about 20 HZ to about 60 HZ for about 40 seconds. In certain embodiments, the one or more pulses can include three pulses (e.g., such that three pulses are sent every 20-60 Hz). Any other suitable power signal for causing the desired effect (e.g., for preventing propagation of a traumatic brain injury) is contemplated herein.

In accordance with at least one aspect of this disclosure, a system can include any suitable embodiment of a wearable article disclosed herein, e.g., as described above. The system can also include an external power supply (e.g., as described above) configured to connect to the wearable article to selectively provide power to the one or more stimulators. In certain embodiments, a manual switch can be disposed between the external power supply and an output connector (e.g., of a cable) and configured to be operated by a user to allow energy to flow from the external power supply to the one or more stimulators.

In certain embodiments, a power control module can be configured to be operatively connected to the one or more sensors to receive data from the one or more sensors and/or to allow energy to flow from the external power supply to the one or more stimulators as a function of the data received from the one or more sensors. In certain embodiments, the power control module can be configured to activate the one or more stimulators when the pressure, the force, or the acceleration are above a shock threshold and/or when the tissue activity is of a predetermined characteristic.

In accordance with at least one aspect of this disclosure, a wearable article can include one or more sensors configured to sense one or more of pressure, force, acceleration, and/or tissue activity, one or more stimulators configured to generate an electric field and/or a magnetic field and positioned to apply the electric field and/or magnetic field to a tissue of a user, and a control module operatively connected to the one or more sensors and the one or more stimulators and configured to activate the one or more stimulators when the pressure, the force, or the acceleration are above a shock threshold and/or when the tissue activity is of a predetermined characteristic. In accordance with at least one aspect of this disclosure, a helmet or helmet padding can be configured to detect and/or treat a traumatic brain injury.

In accordance with at least one aspect of this disclosure, a medical device system configured for the rapid response treatment of a traumatic brain injury in a prehospital environment can include a pad system configured to be applied to a patients head to provide diagnosis and/or treatment of the traumatic brain injury. The medical device system can include any suitable embodiments of a wearable article and/or a system and/or any components thereof as disclosed herein, e.g., as described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a side schematic view of an embodiment of a wearable article in accordance with this disclosure, showing components schematically positioned relative to a shape of the wearable article;

FIG. 2 is a bottom plan view of the embodiment of FIG. 1;

FIG. 3 is a schematic view of an embodiment of a padding including an embodiment of a stimulator in accordance with this disclosure;

FIG. 4 is a schematic diagram of a wearable article in accordance with this disclosure, shown connected to an external power supply;

FIG. 5 is a perspective view of an embodiment of an arrangement of stimulators around a helmet shape in accordance with this disclosure;

FIG. 6 is a cross-sectional view of an embodiment of a helmet in accordance with this disclosure;

FIG. 7 is a front view of an embodiment of a helmet in accordance with this disclosure;

FIG. 8 is a bottom plan view of the embodiment of FIG. 7;

FIG. 9 is a side elevation view of the embodiment of FIG. 7; and

FIG. 10 is a side elevation schematic view of an embodiment of an arrangement of an embodiment of stimulators relative to a brain in accordance with this disclosure;

FIG. 11 is a front elevation schematic view of the embodiment of FIG. 10;

FIG. 12 is a schematic diagram showing an embodiment of a resulting magnetic field output by an embodiment of a stimulator relative to brain tissue in accordance with this disclosure;

FIG. 13 is a chart showing that certain embodiments of treatment in neural tissue results in smaller injury volumes (SRS) when compared to control (SSS); and

FIG. 14 is a fluorescent microscopy image depicting increased blood flow in brain tissue following cTBS treatment.

### DETAILED DESCRIPTION

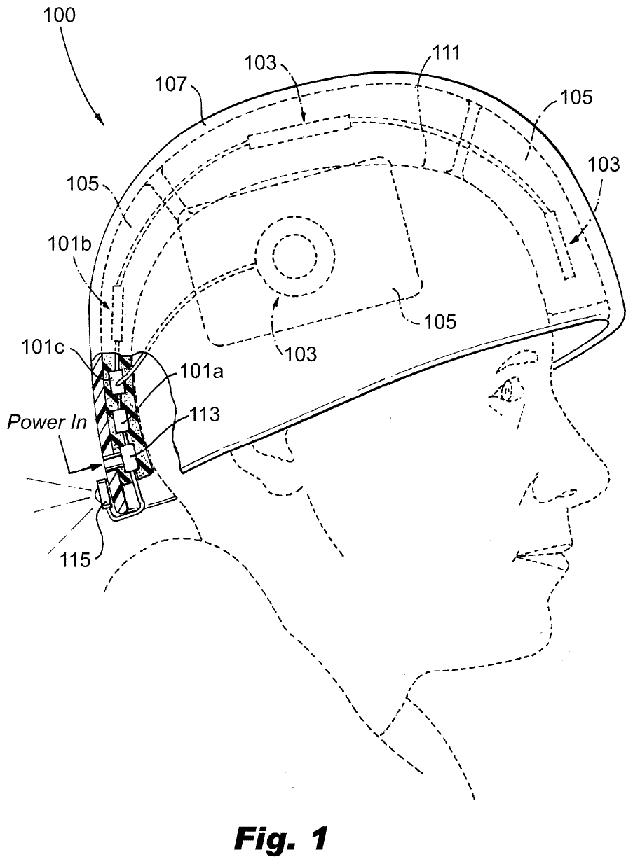

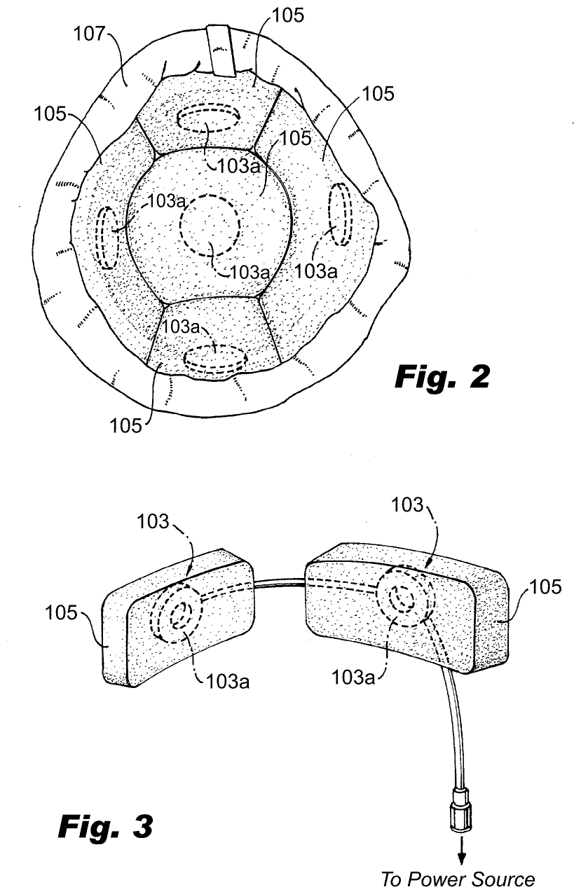

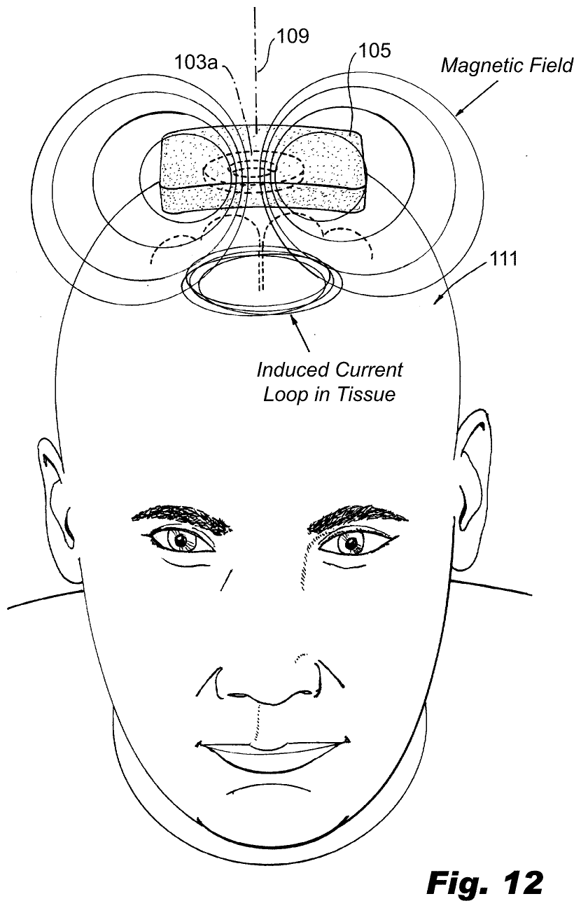

Reference will now be made to the drawings wherein like reference numerals identify similar structural features or aspects of the subject disclosure. For purposes of explanation and illustration, and not limitation, an illustrative view of an embodiment of a wearable article in accordance with the disclosure is shown in FIG. 1 and is designated generally by reference character 100. Other embodiments and/or aspects of this disclosure are shown in FIGS. 2-14. Certain embodiments described herein can be used to treat traumatic brain injuries shortly after injury, for example. Any other suitable use or treatment is contemplated herein.

In accordance with at least one aspect of this disclosure, a wearable article 100 (e.g., a helmet or padding for a helmet) can include one or more sensors 101a, 101b, 101c configured to sense one or more of pressure, force, acceleration, and/or tissue activity. For example, the wearable article 100 can include a pressure sensor 101a, a force sensor 101b, and/or an accelerometer 101c as shown, or any suitable single sensor or combination of sensors configured to sense one or more of pressure, force, acceleration, and/or tissue activity (e.g., brain activity using electroencephalography (EEG)) and/or any suitable combination thereof.

The wearable article 100 can also include one or more stimulators 103 configured to generate a magnetic field and positioned to apply the magnetic field to a tissue (e.g., brain tissue) of a user, i.e., when worn, for treating the tissue after a predetermined pressure, force, acceleration, and/or tissue activity is sensed by the one or more sensors 101a, b, c, for example. For example, after a concussive incident (e.g., a blast, a tackle, a collision), the one or more stimulators 103 can be used to apply a magnetic field to brain tissue quickly to reduce the negative effects of the concussive incident on the brain tissue.

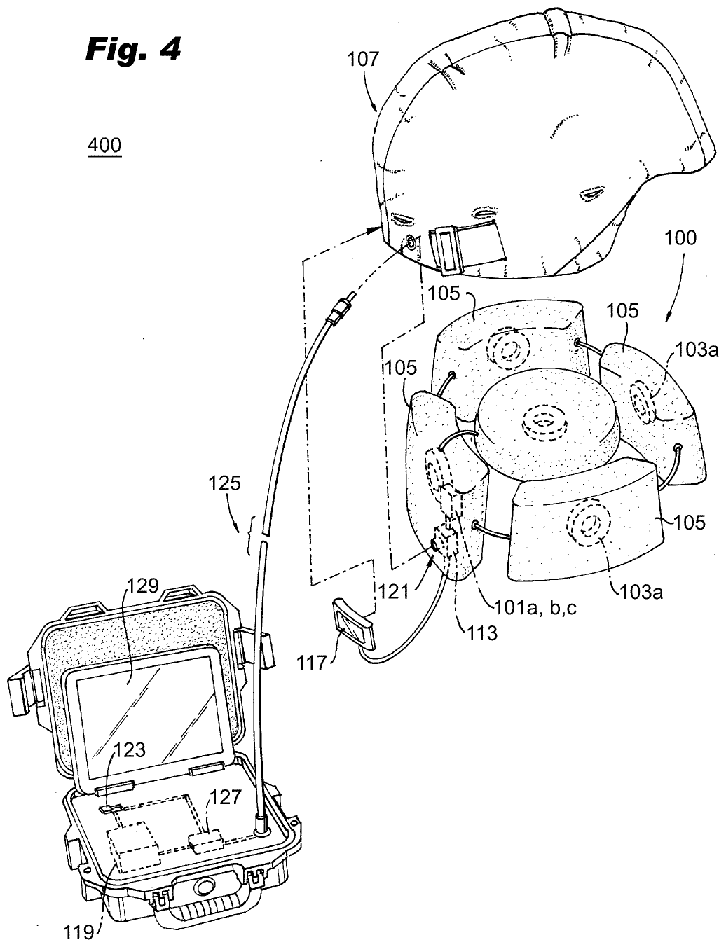

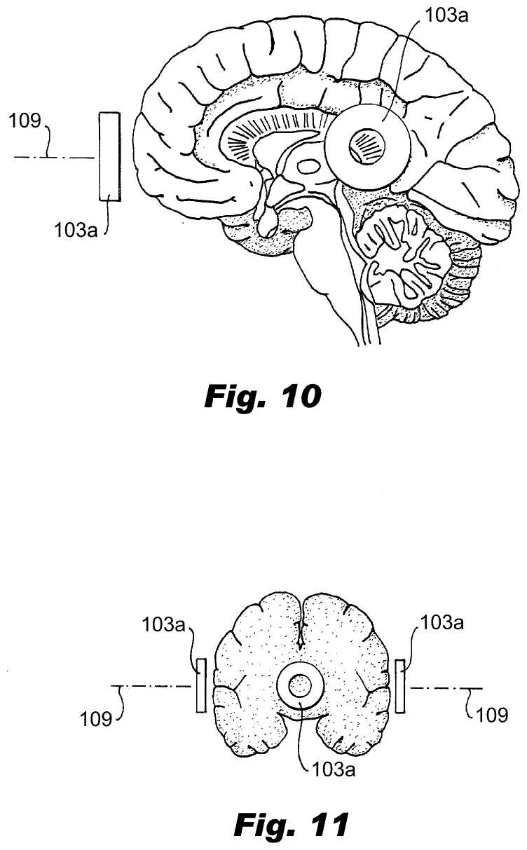

In certain embodiments, each of the one or more stimulators 103 can be configured to apply a respective magnetic field (e.g., as shown in FIG. 10) to brain tissue to prevent propagation of a traumatic brain injury in the brain tissue. For example, referring additionally to FIGS. 2-5, in certain embodiments, the wearable article 100 can be (e.g., as shown in FIGS. 3 and 4) or include helmet padding 105, for example. The one or more stimulators 103 can be disposed in the helmet padding 105 and configured to be positioned to apply the magnetic field to one or more predetermined areas of a brain (e.g., one or more lobes of the brain, all brain lobes, only affected brain lobes, or any suitable desired brain lobes and/or combinations thereof), for example.

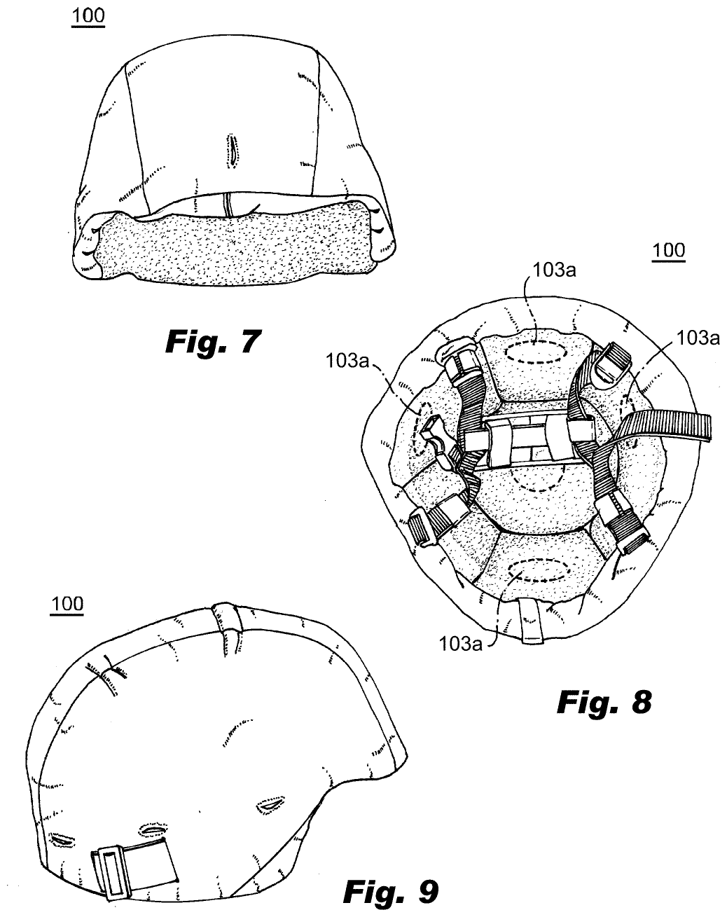

In certain embodiments, the wearable article 100 can include a hard shell 107 (e.g., such that the wearable article 100 is a helmet). The helmet padding 105 (e.g., the array of padding 105 as shown in FIGS. 3, 4, and 5) with the one or more stimulators 103 can be disposed within the hard shell 107, for example. FIG. 6 shows the wearable article 100 being an embodiment of a sports helmet, and FIGS. 7-9 show the wearable article 100 being an embodiment of a combat helmet that can include a padding 105, e.g., as shown in FIGS. 1-5, for example. Any other suitable wearable article (e.g., a shirt, pants, a hat, a band for a limb, a chest covering, etc.) having any suitable components and/or configured for any suitable body part(s) is contemplated herein.

In certain embodiments, e.g., as shown in FIGS. 3 and 4, the one or more stimulators 103 can each include one or more coils 103a. Referring additionally to FIGS. 10, 11, and 12, the one or more coils 103a can be positioned such that a central axis 109 of the one or more coils 103a can be substantially perpendicular (normal) to a scalp surface (e.g., skull 111) when the wearable device 100 is worn. In certain embodiments, the one or more stimulators 103 can be configured to generate a magnetic field having a strength of about 0.5 Tesla (e.g., about 0.5 to about 1 Tesla or above 1 Tesla) and/or above. Any other suitable stimulator (e.g., configured to produce a suitable electric field and/or magnetic field) and/or any other suitable device configured to produce a beneficial treatment to tissue is contemplated herein.

The one or more stimulators 103 (e.g., coils 103a), can be electrically connected in series to each other, in parallel to each other, or not connected to each other, for example. In certain embodiments, certain stimulators 103 can be connected while others are independently powered from other stimulators 103. Any suitable electrical connection for powering the stimulators 103 is contemplated herein. Any suitable wire, material, and/or gauge configured to handle a suitable current to produce a desired magnetic field strength is contemplated herein.

As shown in FIGS. 10, 11, and 12, for example, the one or more stimulators 103 (e.g., coils 103a) can include at least one frontal lobe stimulator and at least one (e.g., two symmetrically opposing) parietal and/or temporal stimulator (e.g., positioned to affect both the parietal and temporal lobe), for example. Any suitable number of stimulators 103 is contemplated herein. Any other suitable stimulator(s) positioned for treating any suitable locations (e.g., occipital lobe, any suitable lobe boundaries) are contemplated herein.

In certain embodiments, the wearable article 100 can include a controller 113 operatively connected to the one or more sensors 101a, b, c and configured to determine when a shock event (e.g., a blast, a concussive force, a collision, a pressure differential between the brain cavity and the atmosphere) indicative of a traumatic brain injury occurs, for example. The wearable article 100 can include a battery 115 operatively connected to the controller 113 and/or the one or more sensors 101a, b, c to provide power to the controller 113 and/or the one or more sensors 101a, b, c for determining if a shock event has occurred.

The wearable article 100 can include an indicator 117 operatively connected to the controller 113, for example. In certain embodiments, the controller 113 can be configured to activate the indicator 117 when the shock event occurs. For example, the indicator 117 can be or include an LED. Any other suitable indicator (e.g., visual, invisible, audible, tactile, etc.) are contemplated herein.

In certain embodiments, e.g., as shown in FIG. 4, the stimulators 103 and/or the controller 113 and/or the one or more sensors 101a, b, c, can be configured to connect to an external power supply 119 (e.g., one or more batteries, capacitors, power electronics, control modules, etc.) via an input 121 (e.g., a coaxial cable). The external power supply 119 can be separate from the wearable article 100, e.g., the helmet, and configured to provide suitable energy to generate each respective magnetic field.

In certain embodiments, however, a suitable power source (e.g., one or more batteries 113 if sized to produce suitable power and/or capacitors) can be connected to and/or contained within the wearable article, e.g., 100 helmet. For example, in certain embodiments, the controller 113 can be configured to cause energy from the power source (e.g., battery 113) to flow to the one or more stimulators 103 to cause generation of each magnetic field.

For example, the controller 113 can be configured to pulse each stimulator 101 to create a pulsed magnetic field. The controller 113 can be configured to provide one or more pulses to each stimulator 103 at a repeating rate of about 20 HZ to about 60 HZ for about 40 seconds. In certain embodiments, the one or more pulses can include three pulses (e.g., such that three pulses are sent every 20-60 Hz). In certain embodiments, power signals to generate theta burst stimulation (cTBS) as appreciated by those having ordinary skill in the art may be used. Any other suitable power signal for causing the desired effect (e.g., for preventing propagation of a traumatic brain injury) is contemplated herein.

In accordance with at least one aspect of this disclosure, as shown in FIG. 4, a system 400 can include any suitable embodiment of a wearable article 100 disclosed herein, e.g., as described above. The system 400 can also include an external power supply 119 (e.g., as described above) configured to connect to the wearable article 100 to selectively provide power to the one or more stimulators 103. The external power supply 119 can include any suitable energy storage device(s) (e.g., one or more batteries, one or more capacitors) configured to output suitable energy to generate the desired electromagnetic field (e.g., as disclosed above). The external power supply 119 can include any suitable power electronics for conditioning output power as needed. In certain embodiments, a manual switch 123 can be disposed between the external power supply 119 and an output connector (e.g., of a cable 125) and configured to be operated by a user to allow energy to flow from the external power supply 119 to the one or more stimulators 103.

In certain embodiments, a power control module 127 can be configured to be operatively connected to the one or more sensors 101a, b, c to receive data from the one or more sensors 101a, b, c and/or to allow energy to flow from the external power supply 119 to the one or more stimulators 103 as a function of the data received from the one or more sensors 101a, b, c. In certain embodiments, the power control module 127 can be configured to activate the one or more stimulators 103 when the pressure, the force, or the acceleration are above a shock threshold and/or when the tissue activity is of a predetermined characteristic. It is contemplated that the power control module 127, the external power supply 119, the switch 123, and any other suitable components (e.g., a display 129 for displaying any suitable data and/or accepting any suitable inputs) can be packaged together (e.g., in a mobile case, e.g., a briefcase, a pelican case, etc.). The control module 127 and the controller 113 can include any suitable hardware and/or software module(s), e.g., as appreciated by those having ordinary skill in the art, configured to perform the disclosed function and/or any other suitable function.

In accordance with at least one aspect of this disclosure, a wearable article 100 can include one or more sensors 101a, b, c configured to sense one or more of pressure, force, acceleration, and/or tissue activity (e.g., brain activity using EEG), one or more stimulators 103 configured to generate an electric field and/or a magnetic field and positioned to apply the electric field and/or magnetic field to a tissue of a user, and a control module 113 operatively connected to the one or more sensors 101a, b, c and the one or more stimulators 103 and configured to activate the one or more stimulators 103 when the pressure, the force, or the acceleration are above a shock threshold and/or when the tissue activity is of a predetermined characteristic. One or more sensors can also be configured to detect abnormal brain activity by EEG in coordination with a treatment algorithm, for example.

In accordance with at least one aspect of this disclosure, a helmet (e.g., as shown in FIGS. 6-9) or helmet padding (e.g., as shown in FIGS. 3 and 4) can be configured to detect and/or treat a traumatic brain injury. In certain embodiments, the helmet and/or padding may not include one or more sensors, and only include one or more stimulators configured to be powered by any suitable local and/or external source. In this regard, in certain embodiments, no logic, diagnosis, or controller may be required where the power supply is external to the helmet or padding for example, an electrical connection to the one or more stimulators can be provided to connect to the external power bank. This can simplify certain embodiments for any suitable use, e.g., for military or sports equipment where data may not be require, a TBI assumed under suitable criteria (e.g., unconscious patient), and treatment initiated. However, any suitable arrangement of sensors, controllers, stimulators, wearable components (e.g., padding, helmet shell), power supplies, cables, etc., is contemplated herein.

In accordance with at least one aspect of this disclosure, a medical device system can be configured for the rapid response treatment of a traumatic brain injury in a prehospital environment. The system can include a pad system (e.g., as shown in FIG. 4) configured to be applied to a patients head to provide diagnosis and/or treatment of the traumatic brain injury. The medical device system can include any suitable embodiments of a wearable article and/or a system and/or any components thereof as disclosed herein, e.g., as described above.

In accordance with at least one aspect of this disclosure, a method can include treating a suspected or known traumatic brain injury patient in a prehospital environment using one or more of a magnetic field and/or electric field. Treatment can include any suitable parameters as appreciated by those having ordinary skill in the art in view of this disclosure, e.g., as disclosed herein, e.g., as described above. Treatment can include using any suitable embodiment of a wearable article and/or a system as disclosed herein, e.g., as described above.

Theta Burst Stimulation (TBS), for example, includes magnetic pulses that are applied in a certain pattern, called bursts. Research studies with TBS have been shown to produce similar if not greater effects on brain activity compared to standard repetitive transcranial magnetic stimulation (rTMS). In certain embodiments, a theta burst pattern can include three bursts of pulses given at 50 Hz and repeated every 200 ms, for example. In certain embodiments, TMS procedures can last up to about 37 minutes per session whereas cTBS can reduce energy application to as little as under a minute, e.g., as disclosed above. However, it is contemplated that embodiments can employ traditional rTMS, cTBS, or any other suitable signal regime desired.

Embodiments can be integrated into a helmet, for example, and can connect to an external diagnostic and treatment unit that can provide the mechanism of action for transcranial magnetic stimulation. In certain embodiments, the one or more sensors and the one or more stimulators (e.g., a stimulator array) can be embedded into the helmet padding such that the helmet padding can be a single, linked, detachable system, for example.

In certain embodiments, the battery, control circuitry, and LED indicators for injury detection can be located externally on the helmet or internally. There can be a computer inside of an external treatment system, which, given sensor input, can apply an algorithm to the data, indicating likelihood of TBI severity to the wearer of the wearable article. After giving this information, the algorithm can prompt the user for Yes or No questions, such as, “Was a traumatic injury witnessed?” and “Did the patient lose consciousness?”, and, if yes to both of these, the system (e.g., the control module) can prompt the user to initiate treatment with TMS or cTBS for example. In certain embodiments, treatment can be applied in the absence of sensor data as well, e.g., using questions that provide after the fact information (e.g., answers to the above two questions) as a predicate for determining a TBI diagnosis.

In certain embodiments, the control module can be configured to autonomously determine if treatment is required and activate treatment based on the sensor data. Embodiments can provide a stand-alone system for the diagnosis and treatment of TBI by even an untrained first responder. In certain embodiments, in addition to or independent of any other sensor types, one or more integrated EEG sensors can be implemented into helmet padding for additional sensing. In certain embodiments, diagnosis of TBI by first responder can be done using with or without sensor data by answering one or more prompted questions in the integrated system, for example.

Treatment with electric fields independent of and/or in conjunction with magnetic fields are contemplated herein. In certain embodiments, a first and second stimulator can be symmetrically placed proximate to both parietal/temporal regions (e.g., above each ear). The arrangement of stimulators can be configured to be hemispherically symmetric around a brain when the helmet is wearable article is worn, for example. Certain embodiments can be reduced to one stimulator for both hemispheres using a stimulator that can create a magnetic field large enough to effect both hemispheres regions.

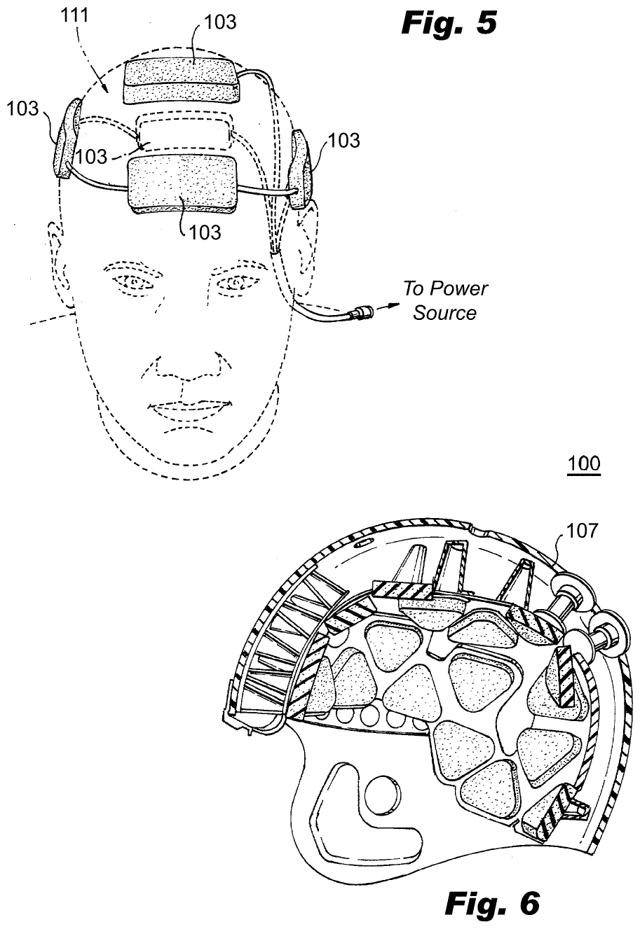

Embodiments can have one or more frontal lobe stimulators (e.g., forehead region), and/or one or more top stimulators (e.g., as shown in FIGS. 1, 2, and 5), and/or one or more occipital lobe stimulators (e.g., as shown in FIG. 5). Any other suitable number of stimulators and positions thereof are contemplated herein.

Certain embodiments can produce a magnetic field of about 0.5 to about 1 Tesla minimum field strength. Stimulators can be or include a coil built around a magnetic core, or butterfly coils, for example, and can be positioned such that the coil axis is pointing at the scalp. In embodiments, the magnetic field can be turned on and off at a certain frequency, e.g., a pulsed frequency, e.g., as in cTBS. In certain embodiments, three pulses of about 50 Hz every about 5 Hz can be applied for about 40 seconds, and then treatment can be done. In certain embodiments, the signal can be pulsed at about 1 Hz constant for about 20 to about 40 minutes after injury.

Certain embodiments that require a strong electric and/or magnetic field can require a significant amount of power. In certain embodiments, e.g., of a helmet, the helmet can include coils, sensors, a controller, and a small battery for powering sensor/logic electronics. A separate treatment system, e.g., a pelican case with a power bank (e.g., large capacitors and/or batteries) can includes logic for diagnosis, sensor readout, and initiation of electric and/or magnetic field application, for example. The helmet can be connected to the pelican case (e.g., via coax cable) to power the coils. In certain embodiments, when a blast happens, the controller can indicate via an LED indicator that a TBI has occurred. A first responder can see indicator, plug the helmet into the treatment system, see and/or evaluate what happened based on the sensor data, then click a button/throw a switch to initiate treatment.

Certain embodiments can include a helmet that senses injury, e.g., implied by exceeding a force threshold, and initiate treatment automatically by activating electric and magnetic fields. Certain embodiments can include a hard exterior, a piezoelectric sensing array that detects force, pressure, acceleration magnitude and direction, a foam padding, a biocompatible electrode contacting scalp configured to produce alternating electric and magnetic fields generated by current manipulation of electrode. A DC hyperpolarizing current waveform can be run through each electrode, effecting neural membrane hyperpolarization. Embodiments can include a Lithium Ceramic Battery, a PCB that registers incoming force data, and applies a current to one or more of the electrodes as desired.

Embodiments can provide a method to prevent neural damage. Embodiments can include a conductive CNT-graphene-carbon aerogel embedded into helmet padding (or any conductive electrode capable of producing electric and magnetic field). Embodiments can include a Force/Acceleration Sensor, computer chip motherboard, flexible battery, and electrodes all housed within padding of helmet, for example. Embodiments can include a helmet that instantly detects a blast and applies electric and/or magnetic fields to the underlying brain to hyperpolarize neurons, causing vast reductions in neural activity, for example.

In certain embodiments, an intelligent blast sensor, that after detection of blast, can send a signal to a processing chip. If force, pressure, or acceleration are above threshold for brain injury, the processing chip can initiates an output current to a downstream biocompatible electrode, which can have wire back to the board to complete the circuit.

An embodiment of a biocompatible electrode can be composed of carbon nanotube graphene cellulose magnetic nanoparticle aerogel that can rest on hairy scalp. Embodiments of an electrode can have parallel aerogel layers, separated by an insulator. The signal output from computer chip can be about 40 Hz or about 0 Hz DC waveform after rectification from AC, for example. The DC current can travel across the electrode and travel to the negative electrode, and no current will run across the scalp due to the far larger conductivity of aerogel relative to the scalp (0.001 ohms vs 10 kiloohms), and the aerogel is waterproof. The constant DC hyperpolarizing current causes as pulsed cathodal electric field, causing the subsequent hyperpolarization of all neurons proximal to the field. Simultaneously at 20-40 Hz, the poles of the aerogel will be switched, causing an alternating magnetic field. This alternating magnetic field can cause the entrainment of deeper areas to activate pro-survival gene transcription in undamaged regions of the brain. In certain embodiments, the electrode can creates an electric field at the surface of the brain, causing the cessation of damaging neural activity, while initiating pro-survival signaling in undamaged, deeper tissue, for example.

An embodiment of an electrode signal can generate a varied biological signal using current manipulation, e.g., capitalizing on a supercapacitive and superconductive biocompatible electrode to create a constant DC hyperpolarizing current field and alternating magnetic field using the same 3D architecture by switching the lateral current output path while maintaining the vertical DC component, for example. Embodiments include using current manipulation within a 3-dimensional superconductor/supercapacitor to intelligently cause specific activation of hyperpolarizing of neurons at different depth after injury. This uses electric and/or magnetic fields to specifically inhibit the disease processes occurring immediately at the point of injury. These fields can actively increase the membrane potentials of neurons and activated secreting microglia, and this technique can both treat neural damage and reduce injury related neuroinflammation.

Embodiment can include using a DC voltage to modify an AC circuit in a 3-dimensional conductor, thus causing stochastic electrical resonance in a pseudo supercapacitor, and manifesting electrical resonance in the 3D superconductor at a specific, desired frequency. Embodiments of a circuit design can induce stochastic resonance in the 3-dimensional supercapacitor comprising an aerogel (e.g., carbon or otherwise), thus increasing the energy input of the capacitor in the AC circuit. The energy absorbed in the supercapacitor can be from the contact of radio waves of same frequency as the AC circuit.

Embodiments can include a method to produce simultaneous electric and magnetic fields by current manipulation in a 3-dimensional soft, compressible, biocompatible, superconducting, supercapacitor. Embodiments can create simultaneous electric and magnetic fields tailored to manipulating specific disease processes in the brain. These fields, either alone or together, when applied by the new stimulus method and electrode system, can inhibit the spread of disease mechanisms while simultaneously activating pro-survival signaling, for example. The proximal electric field in a DC waveform current can inhibit surface neural activity, while an alternating magnetic field can entrain neurons in the deeper undamaged regions by entraining neural activity in deeper gray and white matter to the local resonant frequency. Embodiments can include applying simultaneous electric and magnetic fields to affect neural substructures in a neuroprotective manner. Embodiments can include using flexible, soft, compressible, biocompatible, superconducting electrodes consisting of carbon nanotubes, graphene, or any suitable materials when assembled into a 3-dimensional aerogel. Embodiments can include using DC voltage to induce stochastic coherence resonance in a sensing AC circuit to record underlying neural activity. Embodiments can include using aerogel electrodes to detect impact based off of piezoelectric changes in resistance. Embodiments can include any implementation or form the combination of these principles may result in, for example.

Embodiments can include a method of producing and mass-manufacturing soft bioelectrodes made of CNT graphene (carbon) aerogels. For example, embodiments can include using an induction forge technique under a constant flowing atmosphere of neutral gas to process carbon hydrogel into carbon aerogel. By rapid induction heating, the carbon hydrogel is rapidly converted into carbon aerogel for mass manufacturing purposes.

Embodiments can include a stimulation mechanism for treating disease processes occurring during Traumatic Brain Injury, including but not limited to cardiovascular disease, vascular trauma, and other diseases, for example. By using DC voltage to induce stochastic coherence resonance in the supercapatictive electrode, the AC circuit connected to the electrode can sense small subthreshold neural signals by responding to local electric fields in the brain. Embodiments can provide a way of recording neural stimulation by using a sensor with a stochastic and coherence resonance component.

The disease mechanisms underlying TBI can cause the expansion of initial brain lesions by destroying healthy, undamaged tissue usually within about 1 hour after head injury. Embodiments include a medical device capable of freezing the expansion of brain damage in the prehospital environment, e.g., using rapid-response cTBS technology. Embodiments can allow treatment instantly, and certainly in under 1 hour from trauma.

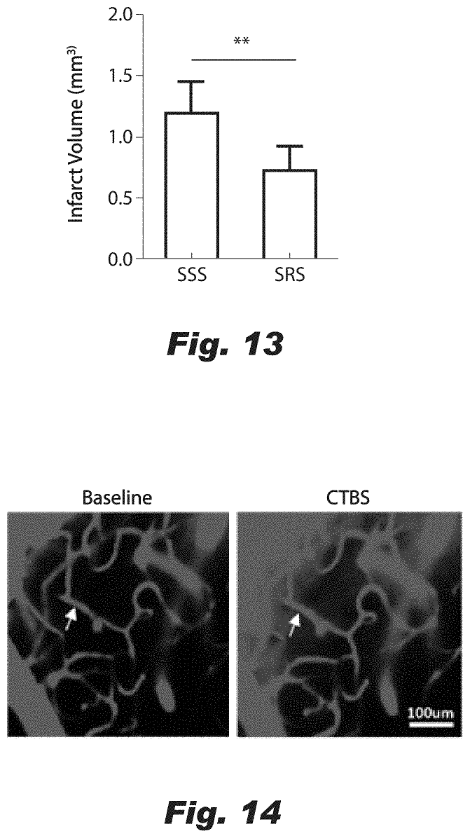

Embodiments can be inserted into a helmet for autonomous treatment initiation or applied by a first responder, for example. Certain embodiments may pose no safety concerns for uninjured patients either, for example. cTBS activates inhibitory neurotransmitter signaling in the cortex causing increased blood flow to the site of injury and decreased inflammatory response, e.g, as shown in FIGS. 13 and 14. Embodiments can be shown to disrupt progression of aberrant excitatory transmission, increased blood flow to the site of injury and disruption of aberrant neurological activity resulting in decreased injury volume, decrease injury volume leading to improved outcomes after TBI, and pose no risk to uninjured patients.

For Traumatic Brain Injury patients, embodiments can minimize brain damage to prevent lifelong disability. Embodiments can be used in any suitable application, e.g., sports, military, first responders, etc.

It is noted that the highest prevalence of TBI is seen in the military. 1 in 4 soldiers experience some form of TBI. Soldiers lack access to any form of neurological protection in the critical 30 minute to one hour window. The highest incidence of TBI in civilian life is seen in emergency medical response. Nearly 2 million dispatched EMS calls are for TBI in the United States annually. There are no current FDA approved treatments for TBI in the prehospital environment. The US Healthcare system spends at least $86 billion per year treating TBI.

Generally, currently, no treatments exist for the treatment of Traumatic Brain Injury (TBI). NeuroVive Pharmaceuticals is developing an intravenous drug for treatment of TBI. However, this drug-based treatment cannot be implemented effectively in the prehospital environment, as only 15.9% of first responders are trained to start an IV. Embodiments provide a noninvasive device-based treatment with distinct advantages as it can be administered by anyone, including an untrained bystanders, and can saves precious minutes in the setting of TBI.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A wearable article, comprising:
one or more sensors configured to sense one or more of a pressure, a force, an acceleration, tissue activity; and
one or more stimulators configured to generate a magnetic field and positioned to apply the magnetic field to a tissue of a user when worn for treating the tissue after a predetermined pressure, force, acceleration, and/or tissue activity is sensed by the one or more sensors or when.

2. The wearable device of claim 1, wherein each of the one or more stimulators are configured to apply a respective magnetic field to brain tissue to prevent propagation of a traumatic brain injury in the brain tissue.

3. The wearable device of claim 2, wherein the wearable article further comprising helmet padding, wherein the one or more stimulators are disposed in the helmet padding and configured to be positioned to apply the magnetic field to one or more predetermined areas of a brain.

4. The wearable device of claim 3, wherein the wearable article further comprises a hard shell, wherein the helmet padding with the one or more stimulators is disposed within the helmet shell.

5. The wearable device of claim 4, wherein the one or more stimulators each include one or more coils, wherein the one or more coils are positioned such that a central axis of the one or more coils is substantially perpendicular to a scalp surface when the wearable device is worn.

6. The wearable device of claim 5, wherein the one or more stimulators are configured to generate a magnetic field having a strength of about 0.5 Tesla or above.

7. The wearable device of claim 6, wherein the one or more stimulators include at least one frontal lobe stimulator and at least one parietal and/or temporal stimulator.

8. The wearable device of claim 6, further comprising a controller operatively connected to the one or more sensors and configured to determine when a shock event indicative of a traumatic brain injury occurs.

9. The wearable device of claim 8, wherein the controller is configured to activate an indicator when the shock event occurs.

10. The wearable device of claim 9, wherein the indicator is or includes an LED.

11. The wearable device of claim 10, wherein the stimulators and/or the controller and/or the one or more sensors are configured to connect to an external power supply via an input, wherein the external power supply is separate from the helmet and configured to provide suitable energy to generate each respective magnetic field.

12. The wearable device of claim 6, further comprising a power source connected to and/or contained within the helmet, wherein the controller is configured to cause energy from the power source to flow to the one or more stimulators to cause generation of each magnetic field.

13. The wearable device of claim 12, wherein the controller is configured to pulse each stimulator to create a pulsed magnetic field.

14. The wearable device of claim 13, wherein the controller is configured to provide one or more pulses to each stimulator at a repeating rate of about 20 HZ to about 60 HZ for about 40 seconds.

15. The wearable device of claim 14, wherein the one or more pulses includes three pulses.

16. A system, comprising:
the wearable article of claim 1, and;
an external power supply configured to connect to the wearable article to selectively provide power to the one or more stimulators.

17. The system of claim 16, further comprising a manual switch disposed between the external power supply and an output connector and configured to be operated by a user to allow energy to flow from the external power supply to the one or more stimulators.

18. The system of claim 16, further comprising a power control module operatively configured to be operatively connected to the one or more sensors to receive data from the one or more sensors and/or to allow energy to flow from the external power supply to the one or more stimulators as a function of the data received from the one or more sensors.

19. The system of claim 18, wherein the power control module is configured to activate the one or more stimulators when the pressure, the force, or the acceleration are above a shock threshold and/or when the tissue activity is of a predetermined characteristic.

20. A wearable article, comprising:
one or more sensors configured to sense one or more of pressure, force, acceleration, and/or tissue activity;
one or more stimulators configured to generate an electric field and/or a magnetic field and positioned to apply the electric field and/or magnetic field to a tissue of a user; and
a control module operatively connected to the one or more sensors and the one or more stimulators and configured to activate the one or more stimulators when the pressure, the force, or the acceleration are above a shock threshold and/or when the tissue activity is of a predetermined characteristic.

21. A helmet or helmet padding configured to detect and/or treat a traumatic brain injury.

22. A medical device system configured for the rapid response treatment of a traumatic brain injury in a prehospital environment, comprising a pad system configured to be applied to a patients head to provide diagnosis and/or treatment of the traumatic brain injury.
